(19) United States
(12) Patent Application Publication
(10) Pub. No.: US 2006/0211684 A1
(43) Pub. Date: Sep. 21, 2006

(54) Use of alpha-2 adrenergic receptor agonists

(75) Inventor: Mark A. McCamish, Cupertino, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Perlegen Sciences, Inc., Mountain View, CA (US)

(21) Appl. No.: 11/331,591

(22) Filed: Jan. 13, 2006

Related U.S. Application Data
(60) Provisional application No. 60/644,255, filed on Jan. 13, 2005.
(60) Provisional application No. 60/651,293, filed on Feb. 8, 2005.

(51) Int. Cl. A61K 31/551; A61K 31/5415; C12Q 1/68

(52) U.S. Cl. 514/220; 514/225.8; 435/6

(57) ABSTRACT

The present invention provides a strategy to compensate for deficiency in the alpha-2C receptor by administering an agonist of different receptors; the alpha-2A and/or dopamine d2 receptors. These receptors are fully functional and receptive to stimulation by an agonist. Agonism of the alpha-2A and/or dopamine d2 receptors by clonidine, Nolomirole or other suitable agonist may down regulate epinephrine production, and hence compensate for the deficiency in the alpha-2C receptor. Such methods are useful for treating a variety of cardiovascular disorders.

### CROSS-REFERENCE TO RELATED APPLICATIONS

The present application is a nonprovisional and claims the benefit of U.S. Ser. No. 60/644,255 filed Jan. 13, 2005 and U.S. Ser. No. 60/651,293 filed Feb. 8, 2005, both incorporated by reference in their entirety for all purposes.

### BACKGROUND OF THE INVENTION

Alpha adrenergic receptors are plasma membrane receptors which are located in the peripheral and central nervous systems throughout the body. They are members of a diverse family of structurally related receptors known as 7-transmembrane receptors and transduce signals by coupling to guanine nucleotide binding proteins (G-proteins). Like other adrenergic receptors, the alpha-2 receptors are activated by endogenous agonists such as epinephrine (adrenaline) and norepinephrine (noradrenaline), and synthetic agonists, which promote coupling to G-proteins that in turn alter effectors such as enzymes or channels.

The alpha adrenergic receptor family of adrenergic receptors (AR) consists of two groups: alpha-1 and alpha-2. Of the alpha-2 group, there are three distinct subtypes denoted alpha-2A, alpha-2B and alpha-2C. The subtypes are derived from different genes, have different structures, unique distributions in the body, and specific pharmacologic properties. For example, whereas alpha-2A is expressed mainly in the CNS, alpha-2C is expressed mainly in the periphery.

A mutation known as Δ322-325 in the alpha-2C adrenergic receptor has been associated with congestive heart failure in blacks. Small et al., NEJM;347:1135-42 (2002) (incorporated by reference). This reference compared the presence of two polymorphisms in black and Caucasian subjects with and without congestive heart failure (CHF). The reference reported that among black subjects those that were homozygous for Δ322-325 were over 5 fold more likely to have CHF. In a review of several historical clinical trials including the MOXCON trial and the BEST trial (Bristow, Circulation. 107:1100-1102 (2003)), it was proposed that the efficacy of bucindolol (a β blocker) might be enhanced by excluding black patients or patients with the Δ322-325 polymorphism from the therapy.

Clonidine is an α2 adrenergic agonist used primarily for the treatment of hypertension (Jarrott et al., Clin. Exp. Pharm. Physiol., 14, 471-479 (1987)). This drug stimulates α2 adrenoreceptors in the vasomotor centers, causing a reduction of sympathetic outflow from the central nervous system. Both cardiac output and peripheral resistance are reduced resulting in a decrease in blood pressure. Higher concentrations cause vasoconstriction by activation of postsynaptic receptors in vascular smooth muscle. However, at therapeutic doses typically used (0.2-0.9 mg/day), the advantages of the drug are counter balanced by certain troublesome side effects including dryness of the mouth, dizziness, sedation, and constipation. At toxic doses, clonidine can cause serious cardiopulmonary instability and central nervous system depression in children and adults.

### SUMMARY OF THE CLAIMED INVENTION

The invention provides methods of prophylaxis or treatment of cardiovascular disease in a patient having or at risk of the disease. The methods comprise determining that the patient has a mutation in an α2C adrenergic receptor or a nucleic acid encoding the same; and administering an effective regime of an agonist of an α2A receptor and/or an agonist of a d2 dopamine receptor to effect prophylaxis or treatment in the patient. In some methods, the patient is homozygous for a Δ322-325 mutation. In other methods, the patient is heterozygous for a Δ322-325 mutation. In some methods, the agonist is clonidine. In some methods, the agonist is Nolomirole. In some methods, the regime comprises administering a daily dosage. In some methods, the administration is oral and the dosage is administered for at least a week at a dosage of less than 0.1 mg/day. In some methods, the administration is oral and the dosage is administered for at least a week at a dosage of less than 0.05 mg/day. In some methods, the dosage is no more than 0.01 mg/day. In some methods, the dosage is administered transdermally via a patch and the dosage is less than 0.1 mg per day. In some methods, the dosage is administered intravenously and the dosage is less than 0.1 mg per day. In some methods, the dosage is higher, for example, between about 0.2 and 0.6 mg per day and up to about 2.4 mg/day. In some methods, the dosage is higher, for example, between about 5-10 mg per day. In some methods, the disease is hypertension. In some methods, the patient has average blood pressure within optimal, normal range or prehypertensive range and has a risk factor of hypertension other than the alpha-2C mutation. In some methods, the patient has average blood pressure greater than systolic 129 mm Hg and/or diastolic 84 mmHg. In some methods, the administering of the agonist reduces the average blood pressure to within normal range (systolic 120-129 mm Hg and diastolic 80-84 mmHg). Some methods further comprise administering a drug other than the agonist to reduce average blood pressure to within normal range.

In some methods, patient's blood pressure is at least 130 systolic and/or 80 diastolic mm Hg. In some methods, the patient's blood pressure is at least 160 systolic and/or 100 diastolic mm Hg. In some methods, the patient's blood pressure is at least 180 systolic and/or 110 diastolic mm Hg. In some methods, the patient has or is at risk of a myocardial disorder. In some methods, the administration is followed by surgery, optionally the surgery is not heart surgery. In some methods, the patient has elevated ambulatory blood pressure but normal blood pressure during examination. In some methods, the patient has an abnormal cardiovascular response to exercise but a normal cardiovascular response at rest. In some methods, the disease is heart failure. In some methods, the patient has or is at risk of hypertension or heart failure, and the administering extends the life of the patient for a period in excess of the mean additional life expectancy for comparable untreated patients. In some methods, the patient has or is at risk of hypertension or heart failure, and the administering improves exercise tolerance or capacity of the patient relative to the tolerance or capacity before administering the agonist. In some methods, the disease is dyspnea. In some methods, the patient has a myocardial disorder selected from the group consisting of myocardial infarction, atrial abnormality, arrhythmia, infection, ventricular hypertrophy, and coronary artery disease. In some methods, the patient has blood pressure within normal range (systolic 120-129 mm Hg and diastolic 80-84 mm Hg).

Some methods further comprise determining a blood pressure for the patient, wherein if the blood pressure is above a predetermined threshold a drug in addition to the agonist is administered to treat the blood pressure. In some such methods, the threshold is a blood pressure of at least 145 systolic and/or 90 diastolic mm Hg. In some such methods, the patient has diabetes or proteinuria and the threshold is a blood pressure of at least 130 systolic and/or 80 diastolic mm Hg.

The invention further provides methods of prophylaxis or treatment of hypertension in a patient having or at risk of hypertension. These methods comprise administering an effective regime of an agonist of an α2A receptor and/or an agonist of a d2 receptor to a patient having a mutation in an α2C adrenergic receptor or a nucleic acid encoding the same. The dosage is administered orally on a daily basis for at least a month, and the daily dosage is less than 0.5 mg/day.

The invention further provides methods of prophylaxis or treatment of symptoms of drug withdrawal. Such methods comprise determining that a patient, who is suffering from or at risk of symptoms of drug withdrawal, has a mutation in an α2C adrenergic receptor or nucleic acid encoding the same, wherein the mutation reduces activity or amount of the receptor expressed from the gene; and administering an effective regime of an agonist of an α2A receptor and/or an agonist of a d2 receptor to effect prophylaxis or treatment in the patient.

The invention further provides methods of anesthetizing a patient. These methods comprise determining that a patient to be anesthetized has a mutation in an α2C adrenergic receptor gene or a nucleic acid encoding the same, wherein the mutation reduces activity or amount of the receptor expressed from the gene; and administering an effective dosage of an agonist of an α2A receptor and/or an agonist of a d2 dopamine receptor to anesthetize the patient.

The invention further provides methods of prophylaxis or treatment of ocular pressure in a patient. These methods comprise determining that a patient, who is suffering from or at risk of a disorder characterized by excess ocular pressure has a mutation in an α2C adrenergic receptor or a nucleic acid encoding the same, wherein the mutation reduces activity or amount of the receptor expressed from the gene; and administering an effective dosage of an agonist of an α2A receptor and/or an agonist of a d2 dopamine receptor to effect prophylaxis or treatment of the disease in the patient.

The invention further provides methods of prophylaxis or treatment of cardiovascular disease in a patient having or at risk of the disease. These methods comprise administering a daily dosage of less than 0.05 mg/day of clonidine for a period of at least a week to a patient to effect prophylaxis or treatment of the disease in the patient, wherein the patient has a mutation in an α2C adrenergic receptor or a nucleic acid encoding the same. In some such methods, the patient is homozygous for a Δ322-325 mutation. In other methods, the patient is heterozygous for a Δ322-325 mutation. In some methods, the patient is administered the daily dosage for at least a month. In some methods, the patient is administered the daily dosage indefinitely. In some methods, the daily dosage is less than 0.01 mg/day.

### BRIEF DESCRIPTION OF THE FIGURE

FIGS. 1 and 2 show that clonidine treatment prevents and reduces hypertrophy and left ventricular systolic pressure in transverse aortic constricted α2C−/−mice. In this model, ventricular contractility is maintained and left ventricular systolic pressures are slightly reduced.

### DEFINITIONS

A risk factor of disease is a genetic, physiologic, clinical, biochemical or other property of a class of individuals that places individuals at a statistically significantly (p≦0.05) higher life-time risk of acquiring the disease than a class of individuals lacking the risk factor.

A patient is at risk of disease, if he or she has at least one risk factor. Usually, the more risk factors a patient has, the greater the risk of disease.

Patient includes humans and other mammals.

The term “clonidine” refers to N-(2,6-dichlorophenyl)-4,5-dihydro-1 H-imidazol-2-amine and includes the pharmaceutically acceptable salts thereof, e.g., the hydrochloride salt thereof. Clonidine is one example of an alpha-2A agonist.

The term “Nolomirole” refers to (±)-5,6,7,8-tetrahydro-6-(methylamino)-1,2-naphthylene diisobutyrate and includes the enantiomers and pharmaceutically acceptable salts thereof, e.g., the hydrochloride salt thereof. Nolomirole is one example of a dopamine d2 receptor agonist. Nolomirole is also an example of an alpha-2A agonist.

The term “pharmaceutically acceptable” means that the ingredient is not a known irritant or sensitizer of human skin or otherwise injurious to a subject and has not been prohibited or restricted from use in topical skin products or other pharmaceuticals by the Food and Drug Administration.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs significantly more frequently, then alleles a and c are in linkage disequilibrium. Linkage disequilibrium may result from natural selection of certain combination of alleles or because an allele has been introduced into a population too recently to have reached equilibrium with linked alleles. (or may be in linkage disequilibrium because very close to one another on a DNA strand (physical proximity))

A marker in linkage disequilibrium can be particularly useful in detecting susceptibility to disease (or other phenotype) notwithstanding that the marker does not cause the disease. For example, a marker (X) that is not itself a causative element of a disease, but which is in linkage disequilibrium with a marker (Y) that is a causative element of a phenotype, can be used detected to indicate susceptibility to the disease in circumstances in which the gene Y may not have been identified or may not be readily detectable.

Allelic variants at the DNA level are the result of genetic variation between individuals of the same species. Some allelic variants at the DNA level that cause substitution, deletion or insertion of amino acids in proteins encoded by the DNA result in corresponding allelic variation at the protein level.

A gene refers to the DNA sequence encoding mRNA and any regulatory sequences, such as promoters, and enhancers present in flanking regions.

A symptom of a disorder means a phenomenon experienced by an individual having the disorder indicating a departure from normal function, sensation or appearance.

A sign of a disorder is any bodily manifestation that serves to indicate presence or risk of a disorder.

A predetermined threshold value is a value of blood pressure, which if met, indicates a particular treatment regimen. Often, the threshold is based on the levels defining different categories of blood pressure in Table 1.

### DETAILED DESCRIPTION OF THE INVENTION

I. General

Patients with the Δ322-325 mutation or other mutation in the alpha-2C receptor gene have a reduced capacity to down regulate epinephrine and consequently experience increased beta-adrenergic responses that may lead to hypertension with predisposition to other diseases of the cardiovascular system. This genetic deficiency cannot be adequately compensated for by treatment with an alpha-2C agonist because the mutant alpha-2C receptor is incapable of stimulation. The present invention provides an alternative strategy to compensate for deficiency in the alpha-2C receptor; that is, by administering an agonist of a different receptor, alpha-2A and/or dopamine d2. These receptors are fully functional and receptive to stimulation by an agonist. Agonism of the alpha-2A receptor by clonidine or other suitable agonist down regulates epinephrine production, and hence compensates for the deficiency in the alpha-2C receptor. Similarly, agonism of the dopamine d2 receptor by Nolomirole or other suitable agonist also down regulates epinephrine production, compensating for the deficiency in the alpha-2C receptor. Because clonidine, Nolomirole, or other alpha-2A and/or d2 agonist is targeted to a subset of patients at risk of or suffering from cardiovascular disease who have the genetic background to benefit from the administration, these drugs can be effective at lower dosages than previously administered, reducing side-effects, e.g., that have plagued prior use of clonidine. Dosages comparable to or higher than prior uses can also be used with reduced side effects due to the defective alpha-2C receptor.

II. Alpha-2A and -2C Genes

The cDNA and amino acid sequences of human alpha adrenergic genes alpha-2A, 2B and 2C are given by Kobilka et al. Science 238, 650-656 (1987); Lomasney et al. Proc.Nat.Acad.Sci. 87, 5094-5098 (1994), and Regan et al. Proc. Natl. Acad. Sci. 85, 6301-6305 (1988) respectively. The proteins have lengths of 450, 450 and 462 amino acids respectively. The genes are located on chromosomes 10, 2 and 4 respectively. The alpha-2C gene is intronless; thus the genomic sequence encoding the alpha-2C receptor is the same as the cDNA (other than flanking regions). The genomic location has been precisely mapped on human chromosome 4p16 near the Huntingdon's disease locus (Riess et al., Genomics 19, 298-302 (1994)). The genomic sequence is at gene 152 of contig NT_{—}006081 of the National Center for Biotechnology Information (NCBI) version NT_{—}006081.17 GI:5146440 (incorporated by reference). The sequence can be obtained at world wide web ncbi.nlm.nih.gov. For present purposes, the sequences in the above cited publications and NT_{—}006081 are regarded as wildtype and sequence variations are viewed as mutants.

A mutation in a nucleic acid encoding alpha-2C means a nucleotide variation (deletion, substitution or addition) relative to the cDNA sequence of Regan et al. Proc. Natl. Acad. Sci. 85, 6301-6305 (1988) or flanking regulatory sequences in genomic DNA as defined by NT_{—}006081. Likewise a mutation in an alpha-2C receptor means a mutation relative to the predicted amino acid sequence of Regan et al., supra. A mutation occurring in the nucleic acid encoding an alpha-2C receptor may or may not result in a mutation in the receptor itself. Preferred mutations are those causing a detectable reduction or loss of function or level of expression of the alpha-2C receptor. Reduced activity can be demonstrated in an assay as described by Small., J. Biol. Chem. 275, 23059-64 (2000) (incorporated by reference). Reduced expression can also be detected at the MRNA level using e.g., a GeneChip® expression monitoring array or at the protein level by immunoassay (e.g., using a ProteinChip® array from Ciphergen). Other mutations in linkage disequilibrium with a mutation causing a loss of function or level of expression can also be used.

An agonist of an alpha-2a receptor means an agonist that agonizes activity of a receptor having the amino acid sequence defined by Lomasney et al. supra.

III. Methods of Detecting Mutations

There are two distinct types of analysis depending whether a mutation in question has already been characterized. The first type of analysis is sometimes referred to as de novo characterization. This analysis compares target sequences in different individuals to identify points of variation, i.e., polymorphic sites. Once polymorphisms have been identified, they can be tested to determine whether they affect the activity or level of expression of the gene in which they occur. Such tests can be performed by association studies (i.e., determining that a polymorphism occurs with increased frequency in individuals having cardiovascular disease). Alternatively, a test can be performed by molecular biology, for example, showing that a cell transformed with a receptor bearing a particular polymorphism has reduced capacity to transducer a signal through the receptor relative to a cell transformed with a wildtype receptor as described by Small et al., US2003/0113725 (incorporated by reference). The second type of analysis is determining which form(s) of a characterized polymorphism are present in individuals under test. There are a variety of suitable procedures for both analyses, which are discussed in turn.

1. Allele-Specific Probes

The design and use of allele-specific probes for analyzing polymorphisms is described by e.g., Saiki et al., Nature 324, 163-166 (1986); Dattagupta, EP 235,726; Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

2. Tiling Arrays

The polymorphisms can also be identified by hybridization to nucleic acid arrays, some example of which are described by WO 95/11995 (incorporated by reference in its entirety for all purposes). One form of such arrays is described in the Examples section in connection with de novo identification of polymorphisms. The same array or a different array can be used for analysis of characterized polymorphisms. WO 95/11995 also describes subarrays that are optimized for detection of a variant forms of a precharacterized polymorphism. Such a subarray contains probes designed to be complementary to a second reference sequence, which is an allelic variant of the first reference sequence. The second group of probes is designed by the same principles as described in the Examples except that the probes exhibit complementarily to the second reference sequence. The inclusion of a second group (or further groups) can be particular useful for analyzing short subsequences of the primary reference sequence in which multiple mutations are expected to occur within a short distance commensurate with the length of the probes (i.e., two or more mutations within 9 to 21 bases).

3. Allele-Specific Primers

An allele-specific primer hybridizes to a site on target DNA overlapping a polymorphism and only primes amplification of an allelic form to which the primer exhibits perfect complementarily. See Gibbs, Nucleic Acid Res. 17, 2427-2448 (1989). This primer is used in conjunction with a second primer which hybridizes at a distal site. Amplification proceeds from the two primers leading to a detectable product signifying the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. The method works best when the mismatch is included in the 3′-most position of the oligonucleotide aligned with the polymorphism because this position is most destabilizing to elongation from the primer. See, e.g., WO 93/22456.

4. Direct-Sequencing

The direct analysis of the sequence of polymorphisms of the present invention can be accomplished using either the dideoxy chain termination method or the Maxam Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

5. Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W.H. Freeman and Co, New York, 1992), Chapter 7.

6. Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target sequences.

7. Single-Base Detection Methods

Single-base extension methods are described by e.g., U.S. Pat. Nos. 5,846,710, 6,004,744, 5,888,819 and 5,856,092. In brief, the methods work by hybridizing a primer that is complementary to a target sequence such that the 3′end of the primer is immediately adjacent to but does not span a site of potential variation in the target sequence. That is, the primer comprises a subsequence from the complement of a target polynucleotide terminating at the base that is immediately adjacent and 5′to the polymorphic site. The hybridization is performed in the presence of one or more labeled nucleotides complementary to base(s) that may occupy the site of potential variation. For example, for a biallelic polymorphisms two differentially labeled nucleotides can be used. For a tetra allelic polymorphisms four differentially labeled nucleotides can be used. In some methods, particularly methods employing multiple differentially labeled nucleotides, the nucleotides are dideoxynucleotides. Hybridization is performed under conditions permitting primer extension if a nucleotide complementary to a base occupying the site of variation in the target sequence is present. Extension incorporates a labeled nucleotide thereby generating a labeled extended primer. If multiple differentially labeled nucleotides are used and the target is heterozygous then multiple differentially labeled extended primers can be obtained. Extended primers are detected providing an indication of which base(s) occupy the site of variation in the target polynucleotide.

8. Protein-based Method

Mutations in alpha-2C receptor can also be detected at the protein level by immunoassay using antibodies known to be specific for particular variants, or by direct peptide sequencing.

IV. Alpha-2A Agonists

An alpha-2A agonist upregulates the alpha-2A receptor. The methods of the invention employ clonidine or another agonist of the alpha-2A receptor. Agonists of alpha-2A can be recognized by their capacity to stimulate phosphorylation in cells transfected with an alpha-2A receptor as described by Small et al., supra. Agonists of the invention may be specific for the alpha-2A receptor (in which case they do not show detectable agonism of alpha-2B and alpha-2C receptors) or may show agonism of alpha-2B and/or alpha-2C as well as alpha-2A. Other available alpha-2A antagonists include:

| | |
|---|---|
| aganodine (Lilly) | guanidine derivative that acts as highly |
| | selective ligand for I₂-imidazole receptor |
| alinidine (Boehringer | N-(2,6-Dichlorophenyl)-4,5-dihydro-N- |
| Ingelheim) | 2-propenyl-1H-imidazol-2-amine 2-(N-allyl- |
| | 2,-6-dichloroanilino)-2-imidazoline 2-[N- |
| | allyl-N-(2,6-dichlorophenyl)amino]-2- |
| | imidazoline (a clonidine analog) |
| benclonidine | 1-Benzoyl-2-(2′,6′-dichlorophenylamino)- |
| (Nycomed Pharma; | 2-imidazoline |
| Norway) | 4,5-Dihydro-1-benzoyl-N-(2,6-dichlorophenyl)- |
| | 1H-imidazol-2-amine |
| fadolmidine | 3-(imidazole-4-ylmethyl)-5-indanol(3RS)-3- |
| (Orion Pharma, | [(1H-imidazol-4-yl)methyl]-2,3-dihydro- |
| Finland) | 1H-inden-5-ol 3-(imidazol-4-ilmetil)-5- |
| | indanol |
| flutonidine | 2-(5-fluoro-o-toluidino)-2-imidazoline |
| Boehringer Ingelheim | hydrochloride |
| | 1H-imidazol-2-amine, 4,5-dihydro-N-(5- |
| | fluoro-2-methylphenyl) monohydrochloride |
| | 2-(2-Methyl-5-fluorophenylamino)-2- |
| | imidazoline hydrochloride |
| idralfidine | antihypertensive compound |
| (Bausch & Lomb) | |
| lofexidine | 2-[1-(2,6-dichlorophenoxy)ethyl]-4,5- |
| (Aventis) | dihydro-1H-imidazole |
| | 2-[1-(2,6-dichlorophenoxy)ethyl]-2- |
| | imidazoline |
| | (structurally related to clonidine) |
| moxonidine | 4-chloro-N-(4,5-dihydro-1H-imidazol-2-yl)- |
| (Lilly) | 6-methoxy-2-methyl-5-pyrimidinamine |
| | 4-chloro-6-methoxy-2-methyl-5-(2-imidazolin- |
| | 2-yl)aminopyrimidine |
| rilmenidine | N-(Dicyclopropylmethyl)-4,5-dihydro-2- |
| (Servier) | oxazolamine |
| | 2-[N-(dicyclopropylmethyl)- |
| | amino]oxazoline |
| | oxaminozoline |
| | α2-Adrenoceptor agonist |
| rolgamidine (Wyeth) | J. Med. Chem., 1985, 28, 1617 |
| Nolomirole (Chiesi | C₁₉H₂₇NO₄.(±)-5,6,7,8- |
| Pharmaceuticals, Inc). | Tetrahydro-6-(methylamino)-1,2- |
| | naphthylene diisobutyrate. |
| | CAS-90060-42-7. INN. |

Random libraries of peptides or other compounds can also be screened for suitability as alpha-2A agonists. Combinatorial libraries can be produced for many types of compounds that can be synthesized in a step-by-step fashion. Such compounds include polypeptides, beta-turn mimetics, polysaccharides, phospholipids, hormones, prostaglandins, steroids, aromatic compounds, heterocyclic compounds, benzodiazepines, oligomeric N-substituted glycines and oligocarbamates. Large combinatorial libraries of the compounds can be constructed by the encoded synthetic libraries (ESL) method described in Affymax, WO 95/12608, Affymax, WO 93/06121, Columbia University, WO 94/08051, Pharmacopeia, WO 95/35503 and Scripps, WO 95/30642 (each of which is incorporated by reference for all purposes). Peptide libraries can also be generated by phage display methods. See, e.g., Devlin, WO 91/18980.

Combinatorial libraries and other compounds are initially screened for suitability by determining their capacity to bind to an alpha-2A receptor. Compounds identified by such screens are then further analyzed for capacity to agonize the receptor by a cellular phosphorylation assay. Several such assays are known. For example, inositol phosphate levels can be measure in confluent CHO cells stably transfected with an alpha-2A receptor incubated with ³H-myoinositol (5 μCi/ml) in media lacking fetal calf serum for 16 hrs at 37° C. in 5% CO₂ atmosphere. Subsequently, cells are washed and incubated with PBS for 30 min followed by a 30 min incubation with 20 mM LiCl in PBS. Cells are then treated with PBS alone (basal), epinephrine alone or epinephrine plus a compound to be screened for agonism for 5 min, and inositol phosphates extracted as described by Martin J. Biol. Chem. 258, 14816-14822 (1983). Following separation on Agl-X8 columns, total inositol phosphates are eluted with a solution containing 0.1 M formic acid and 1 M formate. Agonism is shown by increased inositol phosphates in the presence of agonist plus epinephrine relative to ephinephrine alone.

V. Dopamine 2D Receptor Agonists

The dopamine receptors are a class of G-protein coupled receptors with dopamine as their endogenous ligand. The d2 receptor is negatively coupled to adenylate cyclase via an inhibitory G protein. The cDNA and amino acid sequence are as described by Grandy et al., PNAS 86, 9762-6 (1989). Nolomirole is not only an α2A agonist, but is also dopamine receptor (d2) agonist. A d2 agonist upregulates the d2 dopamine receptor. Recent evidence suggests that the beneficial effect that Nolomirole has on blood pressure may be due to its function as a d2 agonist rather than as an α2A agonist. This observation can be explained in that the d2 and α2A receptors are both presynaptic receptors that have similar functions. Specifically, when stimulated both receptors inhibit secretion of norepinephrine. Norepinephrine is an inotropic agent that increases the frequency and strength of heart muscle contractions and causes the constriction of blood vessels, thereby increasing blood pressure. Therefore, an α2C mutation/deletion can be treated with one or more d2 receptor agonists (e.g., ibopamine (Henwood, Drugs 36, 11-31), quinpirole (Drug Metab. Dispos. 1987 Jan-Feb;15(1):107-13), iodosulpride (Martres et al., Science. May 10;228(4700):752-5 (1985)), bromocriptine (Ergotaman-3′,6′, 18-trione,2-bromo-12′-hydroxy-2′-(1-methylethyl)-5′-(2-methylpropyl)-,(5′α) monomethanesulfonate), ropinirole (hydrochloride salt of 4-[2-(dipropylamino)ethyl]-1,3-dihydro-2H-indol-2-one monohydrochloride) and pramipexole (Miraxpex® Pfizer) and pharmaceutically acceptable salts of any of these), either instead of or in addition to one or more α2A receptor agonists. Other d2 agonists can be identified by screening compounds as described above on cells transfected with a d2 receptor and a CRE-SEAP (cyclic-AMP response elements-secreted alkaline phosphatase) reporter plasmid as described by Durocher et al., Analytical Biochemistry 284, 316-326 (2000)). Drugs that possess both α2A and d2 agonist properties, such as Nolomirole, can be particularly useful for treatment of individuals as described herein.

VI. Patients Amenable to Treatment

Any patient who is identified as having a heterozygous or homozygous mutation in the alpha-2C adrenergic receptor gene, which impairs receptor function or expression or is in linkage disequilibrium with such a mutation, is at risk of cardiovascular disease relative to the general population and is a candidate for treatment with an agonist of alpha-2A and/or d2 receptors. Patients having a homozygous mutation are particularly at risk. Patients having a heterozygous or homozygous mutation in the alpha-2C adrenergic receptor gene may or may not be hypertensive. Patients having at least 1, 2, 3 or 4 or more other risk factors of cardiovascular disease or clinical symptoms are particularly suitable candidates for treatments. Risk factors of cardiovascular disease include high cholesterol level, cigarette smoking, diabetes, family history of heart disease, inactive lifestyle, obesity, proteinuria, African-American race, and male gender. Undergoing surgery whether or not the surgery is associated with the cardiovascular system is also a risk factor for cardiovascular disease. Biochemical markers such as elevated concentrations of natriuretic peptides or protein C are both risk factor of future disease and signs of present cardiovascular disease. Likewise structural abnormalities of the heart detectable by ultrasound or MRI are risk factors and signs of heart disease. Functional abnormalities such as high heart rate or arrhythmias are also risk factors and signs of cardiovascular disease. Other examples of risk factors and treatment strategies are discussed, for example, in Douglas, et al. (2003) “Management of High Blood Pressure in African Americans”, Arch. Intern. Med. 63:525-541.

| | Ranges for Most Adults |
|---|---|
| Blood Pressure Category | (systolic/diastolic) |
| Optimal Blood Pressure | Systolic below 120 mm Hg |
| (Systolic/Diastolic) | Diastolic below 80 mm Hg |
| Normal Blood Pressure | Systolic 120 to 129 mm Hg |
| | Diastolic 80 to 84 mm Hg |
| High Normal Blood Pressure | Systolic 130 to 139 mm Hg |
| (Prehypertension) | Diastolic 85 to 89 mm Hg |
| Hypertension | Systolic at or above 140 mm Hg |
| (High Blood Pressure) | Diastolic at or above 90 mm Hg |
| | (In middle age and older people, |
| | systolic pressure at or above |
| | 140 mm Hg suggests higher |
| | health risks even when diastolic |
| | pressure is normal or low.) |
| Mild Hypertension | Systolic 140 to 159 mm Hg |
| (Stage 1) | Diastolic 90 to 99 mm Hg |
| Moderate Hypertension | Systolic 160 to 179 mm Hg |
| (Stage 2) | Diastolic 100 to 109 mm Hg |
| Severe Hypertension | Systolic 180 to 209 mm Hg |
| (Stage 3) | Diastolic 110 to 119 mm Hg |
| Very Severe Hypertension | Systolic greater than 210 mm Hg |
| (Stage 4) | Diastolic greater than 120 mm Hg |

An individual is classified into the category that corresponds to their “worst” pressure reading, whether systolic or diastolic. For example, a person who has a normal systolic blood pressure (SBP) of 125 mm Hg and a mildly hypertensive diastolic blood pressure (DBP) of 95 would be classified as mildly hypertensive.

In some methods, the blood pressure of an individual is determined before treatment, and optionally monitored throughout treatment. Individuals having blood pressure classified as hypertensive or worse are candidates for treatment. Individuals having above normal blood pressure are at risk of hypertension and are candidates for prophylaxis and treatment. Some individuals have average blood pressure within normal range but show abnormal peaks for short periods or in response to exercise, or when ambulatory. These individuals are also candidates for prophylaxis and treatment to reduce or eliminate the abnormal peaks. Patients who are not hypertensive but who have high normal blood pressure (i.e., are prehypertensive) and a mutation in the alpha-2C adrenergic receptor gene are also candidates for prophylaxis to inhibit development of higher blood pressure and treatment to lower their blood pressure to normal or optimal levels. Individuals having a high normal blood pressure (prehypertensive) and a risk factor for hypertension (e.g., diabetes, proteinuria, left ventricular hypertrophy) as well as an alpha 2C adrenergic receptor gene mutation are candidates for prophylaxis or treatment. Individuals with the alpha 2C deletion are candidates for prophylactic treatment even with normal blood pressure, particularly if one or more other risk factors of cardiovascular disease are present.

Individuals having myocardial disease with or without abnormal blood pressure are candidates for treatment. Examples of such disease include heart failure, myocardial infarction, atrial abnormality, arrhythmia, infection, ventricular hypertrophy, coronary artery disease, stable and unstable angina, nocturnal dyspnea, exercise intolerance and dyspnea on exertion. For example, left ventricular hypertrophy (LVH) is a risk factor of cardiovascular disease, and in particular is a surrogate of coronary heart disease mortality (Brown, et al., Am Heart J 140(6):848-56 (2000); Sundström, et al. Circulation 103:2346 (2001)). Individuals having LVH with or without abnormal blood pressure are candidates for treatment, and such treatment can reverse the hypertrophy in the individual and/or reduce their risk of coronary heart disease mortality. Further, individuals at risk of developing LVH (e.g., having cardiovascular disease (e.g., coronary heart disease), hypertension, obesity, aortic valve stenosis or obstructive cardiomyopathy) are candidates for treatment, and such treatment can prevent the development of LVH. Individuals having or at risk of other diseases of the cardiopulmonary system, such as dyspnea and pulmonary hypertension, are also candidates for treatment.

Agonists of alpha-2A receptor, the dopamine d2 receptor, or both (e.g., Nolomirole) can also be used in individuals having a heterozygous or homozygous mutation in alpha-2C for other indications. Candidates for treatment include individuals who smoke or are addicted to drugs, and who are undergoing or about to undergo a reduction or cessation of tobacco or drug usage (see, e.g., U.S. Pat No. 4,312,878). Clonidine eases symptoms of withdrawal. Other candidates for treatment are individuals undergoing surgical procedures in need of anesthesia (Filos et al., Anesthesiology 81, 591-601 (1994)). Other candidates are individuals suffering or at risk of abnormally high ocular pressure (see, e.g., U.S. Pat No. 5,212,196).

Optionally, an agonist of alpha-2A receptors and/or the d2 receptor can be used in combination with a second drug effective to treat cardiovascular disease, and particularly hypertension. The second drug may or may not have been approved by the FDA or similar body for treatment for hypertension. For example, the second drug can be another agonist of alpha-2A and/or d2 receptors, a β-blocker, an ACE (angiotensin-converting enzyme) inhibitor, a diuretic (e.g., a thiazide diuretic, potassium-sparing diuretic or loop diuretic), an ARB (angiotensin II receptor blocker), or a CCB (calcium channel blocker). Combination treatment can be used for prophylactic or therapeutic applications. Whether an α2A and/or d2 agonist is administered only or in combination with another drug can depend on the intensity of symptoms and number of risk factors of the patient. For example, in some methods, for an individual having an α2C mutation and other risk factors of cardiovascular disease (particularly diabetes or proteinurea), the blood pressure at which administration of a second drug is indicated is lower than that of a first individual who has an α2C mutation but lacks one or more of the additional risk factors. As an example, for an individual who has a systolic blood pressure of at least 145 and/or a diastolic blood pressure of at least 90 and has a hypertension-related risk factor (e.g., diabetes) as well as an alpha 2C receptor gene mutation, a regimen of two drugs rather than one can be initially administered to lower the individual's blood pressure quickly. Such a blood pressure threshold may be lower (e.g., 130/80) if the individual possesses one or more additional risk factors, for example, proteinuria. For a different individual, not having diabetes or proteinurea, but having prehypertensive blood pressure and an alpha2C mutation, only the alpha-2A agonist or d2 agonist may be indicated. For another individual not having diabetes or proteinuria but having the alpha-2C mutation and a blood pressure that is above a certain threshold (e.g., a systolic blood pressure of at least 155 and/or a diastolic blood pressure of at least 100), then the initial treatment recommended may be a regimen of two drugs simultaneously rather than one drug alone as a means of bringing down the blood pressure of the individual more quickly. In other examples, an individual with an alpha 2C deletion has increased risk and should be treated with an alpha 2A agonist or d2 agonist when they are considered to experience pre-hypertension (BP, e.g., a systolic blood pressure>130/85) or even when they have normal blood pressure (e.g., a diastolic blood pressure of>80). Additionally, those individuals with an alpha 2C deletion are at sufficient risk that they can be treated with two agents (an alpha 2A agonist and one other agent) when they are considered to experience hypertension (e.g., a systolic blood pressure>145 and/or a diastolic blood pressure of>90). Examples of drugs that may be used in combination with an agonist of alpha 2A and/or d2 receptors are described in, e.g., Chobanian, et al. (2003) Hypertension 42:1206-1252, which is incorporated herein by reference for all purposes.

VII. Methods of Treatment

In prophylactic applications, an alpha-2A and/or d2 agonist is administered to a patient susceptible to, or otherwise at risk of, a particular disease, as explained above, in an amount sufficient to eliminate or reduce the risk or delay the onset of the disease. In therapeutic applications, an alpha-2A and/or d2 agonist is administered to a patient suspected of, or already suffering from such a disease, as explained above, in an amount sufficient to cure, or at least partially arrest, the symptoms of the disease and its complications. Treatment can, for example, extend the time when hypertension or LVH would usually develop or extend the life of a patient significantly beyond the mean of comparable untreated patients. Treatment can also increase the tolerance or capacity of the patient to exercise. Treatment can also lower average blood pressure from, e.g., hypertensive or prehypertensive range to normal or optimal ranges. An amount adequate to accomplish treatment is defined as a therapeutically- or prophylactically-effective dose, and a regime of amount and frequency adequate to accomplish this is referred to as a therapeutically- or prophylactically-effective regime. In both prophylactic and therapeutic regimes, the alpha-2A and/or d2 agonist is usually administered in several dosages until at least one risk factor, sign or symptom of the disease stabilizes, reduces or disappears. In some methods, the alpha-2A and/or d2 agonist is administered until average blood pressure returns to normal or optimal levels. In some patients, treatment is continued for life.

Effective doses of the compositions of the present invention, for the treatment of the above described conditions vary depending upon many different factors, including means of administration, target site, physiological state of the patient, whether the patient is human or an animal, other medications administered, and whether treatment is prophylactic or therapeutic. Agonists can be administered by a variety of routes such as parenteral, topical, intravenous, oral, subcutaneous, intraperitoneal, intranasal or intramuscular with oral, transdermal via a patch and intravenous being preferred.

Preferred agonists of alpha-2A include clonidine and Nolomirole. Clonidine can be administered orally, transdermally via a patch or intravenously. Previously, clonidine has been administered at a daily dosage of 0. 1-0.3 mg/day, or 0.2-0.6 mg/day up to a maximum of 2.4 mg/day (Anderson, et al. (2002) Handbook of Clinical Drug Data, 10ₜₕ Edition. McGraw-Hill). In the present methods, clonidine can be administered within this range or higher, but is preferably administered at a lower dosage to reduce side effects. Exemplary dosages ranges are less than 0.1 mg per day, less than 0.05 mg/day or less than 0.01 mg. per day. For example, a suitable dosage range is 0.005-0.09 mg/day. Other agonists can be administered at the same dosages by weight or by molarity of active agent, or can be modified by a correction factor reflecting the relative abilities of clonidine and the other agent to agonize the alpha-2A and/or d2 receptors. For example, Nolomirole, which has previously been used at dosages from 5-10 mg/day, can be dosed at 5mg twice a day to achieve similar results. Treatment is continued usually on a daily basis, either indefinitely, or until there is some change in the patient's symptoms or risk factors that indicate that treatment should be modified or discontinued.

Clonidine is commercially available in a weekly transdermal patch (Catapres TTS: 0.1 mg, 0.2 mg, or 0.3 mg/d, with each patch containing 2.5 mg, 5 mg, and 7.5 mg of clonidine, respectively) and in tablet form (Catapres: 0.1 mg, 0.2 mg, and 0.3 mg; Combipres includes 15 mg of chlorthalidone diuretic). Clonidine is rapidly absorbed from the gastrointestinal tract and has excellent CNS penetration because of lipid solubility. Peak plasma concentrations are reached 3-5 hours after a single oral dose. Dermal application may take several days for steady state levels. Plasma half-life is 12-16 hours, with antihypertensive effects occurring within 30-60 minutes of ingestion. Clonidine is excreted unchanged in the urine and metabolized by the liver.

The inactive ingredients in Catapres are colloidal silicon dioxide, corn starch, dibasic calcium phosphate, FD&C Yellow No. 6, gelatin, glycerin, lactose, magnesium stearate, methylparaben, propylparaben. The Catapres 0.1 mg tablet also contains FD&C Blue No. 1 and FD&C Red No. 3. For treating high ocular pressure, an ophthalmic solution is occasionally used in the treatment of glaucoma. A suitable ophthalmic solution containing clonidine or other alpha-2A agonist at a concentration of the agent of from about 0.1% to about 1% by weight, and pH5.6-7.

Other pharmaceutical carriers are described by Remington: The Science and Practice of Pharmacy, Alfonso R. Gennaro, editor, 20th ed. Lippingcott Williams and Wilkins: Philadelphia, PA, 2000. A carrier must be acceptable in the sense of being compatible with the other ingredients of the formulation and not injurious to the subject. Examples of materials which can serve as pharmaceutically-acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; lycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

For oral therapeutic administration, the composition can be combined with one or more carriers and used in the form of ingestible tablets, buccal tablets, troches, capsules, elixirs, suspensions, syrups, wafers, chewing gums, foods and the like. Also, for oral consumption the active ingredient can be dissolved or suspended in water or other edible oral solutions. Such compositions and preparations should contain at least 0.1% of active compound. The percentage of the compositions and preparations can be varied and can conveniently be between about 0.1 to about 100% of the weight of a given unit dosage form. The amount of active agent in such therapeutically useful compositions is such that an effective dosage level is obtained.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame; or a flavoring agent such as peppermint, oil of wintergreen, or cherry flavoring. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac or sugar and the like.

Agonists can be formulated as a sustained release preparation. Numerous techniques for formulating sustained release preparations are described in the following references—U.S. Pat. Nos. 4,891,223; 6,004,582; 5,397,574; 5,419,917; 5,458,005; 5,458,887; 5,458,888; 5,472,708; 6,106,862; 6,103,263; 6,099,862; 6,099,859; 6,096,340; 6,077,541; 5,916,595; 5,837,379; 5,834,023; 5,885,616; 5,456,921; 5,603,956; 5,512,297; 5,399,362; 5,399,359; 5,399,358; 5,725,883; 5,773,025; 6,110,498; 5,952,004; 5,912,013; 5,897,876; 5,824,638; 5,464,633; 5,422,123; and 4,839,177; and WO 98/47491.

For administration by inhalation, the active compound(s) can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

In addition to the formulations described previously, the compounds can also be formulated as a depot preparation. Such long acting formulations can be administered by implantation or transcutaneous delivery (for example subcutaneously or intramuscularly), intramuscular injection or a transdermal patch. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

### EXAMPLE

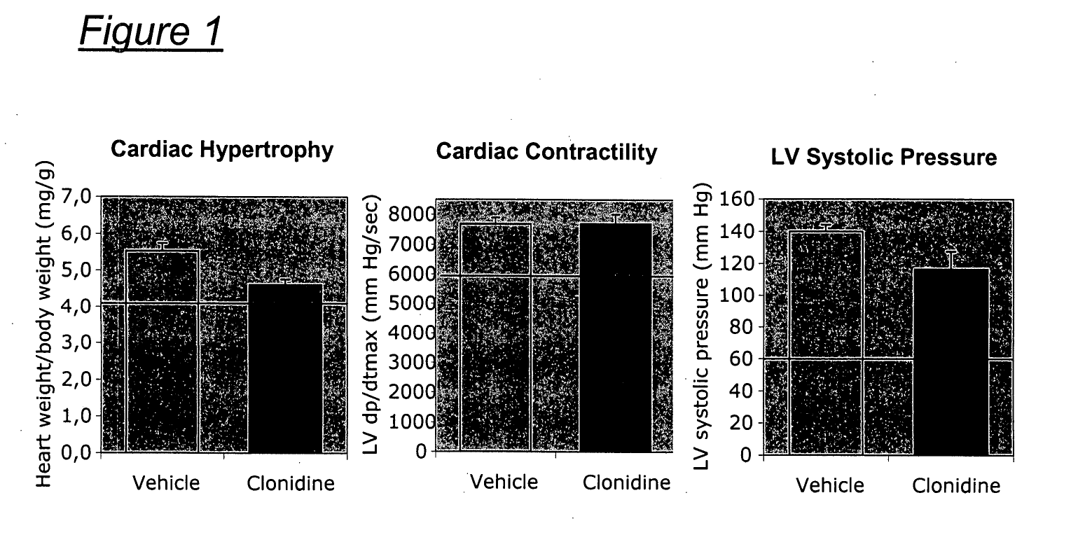

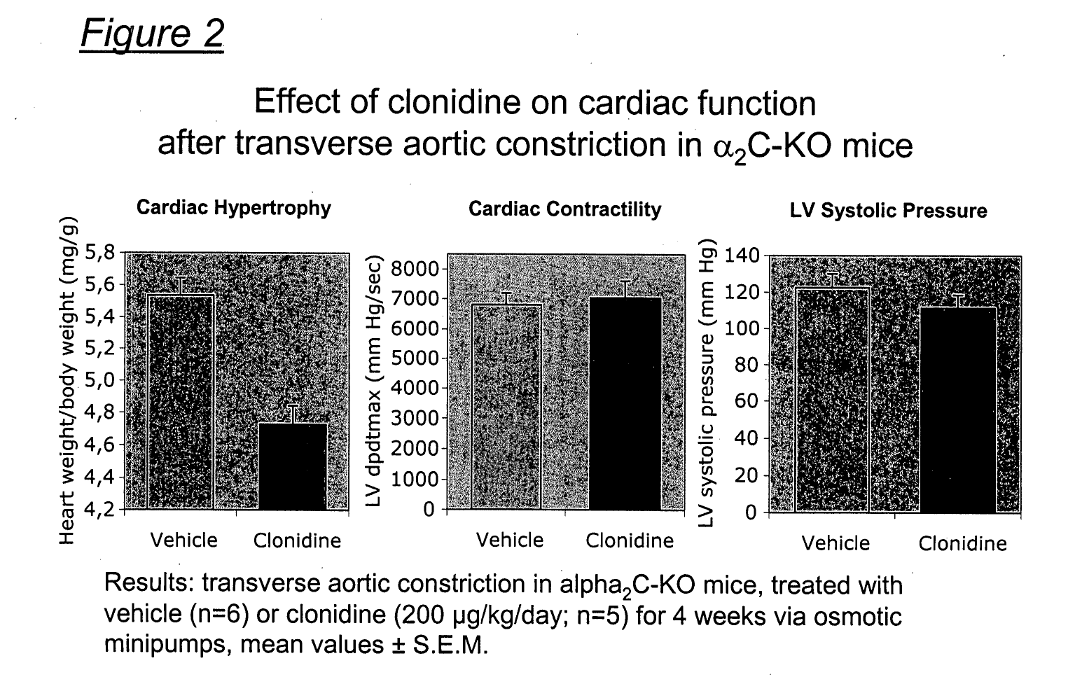

This example tests whether treatment of α₂C−/−knockout transgenic mice with the partial α₂A agonist, clonidine, improves cardiac function and reduces ventricular hypertrophy after left ventricular pressure overload. In other words, the example tests whether clonidine-mediated activation of α_{2A}-receptors compensates for the loss of functional α₂C. The mice used in this experiment are described in more detail in Brede et al., Circulation 106, 2491-2496 (2002). Transverse aortic constriction was applied to induce left ventricular pressure overload, also as described in Brede et al. Mice treated in this way have increased mortality and left ventricular hypertrophy. Mice were treated with 200 μg/kg/day clonidine or vehicle for four weeks. Three mice were treated with clonidine and three with vehicle in the initial experiment. The results are shown in FIG. 1. In brief, treatment with clonidine reduced ventricular hypertrophy as well as left ventricular systolic pressure, but did not effect the contractility of the heart.

Similar results were obtained in a second experiment in which α_{2C} −/−knockout transgenic mice were treated with vehicle (n=6) or clonidine (200 μg/kg/day; n=5) for 4 weeks via osmotic minipumps, mean values ± S.E.M. Again, clonidine reduced ventricular hypertrophy as well as left ventricular systolic pressure, but did not effect the contractility of the heart.

It is apparent from the foregoing that the invention includes a number of uses. The uses include the use of an agonist of an α2A receptor and/or an agonist of a d2 receptor in the manufacture of a medicament to effect prophylaxis or treatment of a cardiovascular disease in a patient determined to have a mutation in an α2C adrenergic receptor or nucleic acid encoding the same. The invention also includes the use of an agonist of an α2A receptor and/or an agonist of a d2 receptor in the manufacture of a medicament to effect prophylaxis or treatment of a cardiovascular disease in a patient when the daily dosage is less than or equal to 10 mg/day and preferably less than 0.5 mg/day, and more preferably less than 0.05 mg/day, optionally for a period of at least a week. The invention also includes the use of an agonist of an α2A receptor and/or an agonist of a d2 receptor in the manufacture of a medicament to effect prophylaxis or treatment of hypertension or ocular pressure in a patient determined to have a mutation in an α2C adrenergic receptor or nucleic acid encoding the same. The invention also includes the use of an agonist of an α2A receptor and/or an agonist of a d2 receptor in the manufacture of a medicament to anesthetize a patient determined to have a mutation in an α2C adrenergic receptor or nucleic acid encoding the same.

It will be apparent to one of ordinary skill in the art that many changes and modifications can be made thereto without departing from the spirit or scope of the appended claims. All publications, patents and patent applications mentioned in this specification are herein incorporated by reference in their entirety for all purposes to the same extent as if each individual publication, patent or patent application was specifically and individually indicated to be incorporated by reference. Unless otherwise apparent from the context, any embodiment, step, feature, element or aspect of the invention can be used in combination with any other.

1. A method of prophylaxis or treatment of cardiovascular disease in a patient having or at risk of the disease, comprising:
determining that the patient has a mutation in an α2C adrenergic receptor or a nucleic acid encoding the same; and
administering an effective regime of an agonist of an α2A receptor and/or an agonist of a d2 dopamine receptor to effect prophylaxis or treatment in the patient.

2. The method of claim 1, wherein the patient is homozygous for a Δ322-325 mutation.

3. The method of claim 1, wherein the patient is heterozygous for a Δ322-325 mutation.

4. The method of claim 1, wherein the agonist is clonidine.

5. The method of claim 1, wherein the regime comprises administering a daily dosage.

6. The method of claim 5, wherein the administration is oral and the dosage is administered for at least a week at a dosage of less than 0.1 mg/day.

7. The method of claim 5, wherein the administration is oral and the dosage is administered for at least a week at a dosage of less than 0.05 mg/day.

8. The method of claim 5, wherein the dosage is no more than 0.01 mg/day.

9. The method of claim 5, wherein the dosage is administered transdermally via a patch and the dosage is less than 0.1 mg per day.

10. The method of claim 5, wherein the dosage is administered intravenously and the dosage is less than 0.1 mg per day.

11. The method of claim 5, wherein the dosage is between about 0.1-2.4 mg per day.

12. The method of claim 5, wherein the dosage is between about 0.3-0.6 mg per day.

13. The method of claim 1, wherein the agonist is Nolomirole.

14. The method of claim 13, wherein the regime comprises administering a daily dosage.

15. The method of claim 13, wherein the administration is oral and the dosage is administered for at least a week at a dosage of between about 5 and 10 mg per day.

16. The method of claim 13, wherein the dosage is administered transdermally via a patch and the dosage is between about 5 and 10 mg per day.

17. The method of claim 13, wherein the dosage is administered intravenously and the dosage is between about 5 and 10 mg per day.

18. The method of claim 1, wherein the disease is hypertension disease, heart failure, ventricular hypertrophy, or dyspnea.

19. The method of claim 18, wherein the patient has average blood pressure within optimal, normal range or prehypertensive range and has a risk factor of hypertension other than the alpha-2C mutation.

20. The method of claim 19, wherein the patient has average blood pressure greater than systolic 129 mm Hg and/or diastolic 84 mmHg.

21. The method of claim 19, wherein the administering of the agonist reduces the average blood pressure to within normal range (systolic 120-129 mm Hg and diastolic 80-84 mmHg)

22. The method of claim 19, further comprising administering a drug other than the agonist to reduce average blood pressure to within normal range.

23. The method of claim 19, wherein the patient's blood pressure is at least 130 systolic and/or 85 diastolic mm Hg.

24. The method of claim 19, wherein the patient's blood pressure is at least 160 systolic and/or 100 diastolic mm Hg.

25. The method of claim 19, wherein the patient's blood pressure is at least 180 systolic and/or 110 diastolic mm Hg.

26. The method of claim 19, wherein the patient has or is at risk of a myocardial disorder.

27. (canceled)

28. (canceled)

29. The method of claim 1, wherein the patient has elevated ambulatory blood pressure but normal blood pressure during examination.

30. The method of claim 1, wherein the subject has an abnormal cardiovascular response to exercise but a normal cardiovascular response at rest.

31. (canceled)

32. (canceled)

33. The method of claim 1, wherein the patient has or is at risk of hypertension or heart failure, and the administering extends the life of the patient for a period in excess of the mean additional life expectancy for comparable untreated patients.

34. The method of claim 1, wherein the patient has or is at risk of hypertension or heart failure, and the administering improves exercise tolerance or capacity of the patient relative to the tolerance or capacity before administering the agonist.

36. The method of claim 1, wherein the patient has a myocardial disorder selected from the group consisting of myocardial infarction, atrial abnormality, arrhythmia, infection, ventricular hypertrophy, and coronary artery disease.

37. The method of claim 36, wherein the patient has blood pressure within normal range (systolic 120-129 mm Hg and diastolic 80-84 mm Hg).

38. The method of claim 1, further comprising determining a blood pressure for the patient, wherein if the blood pressure is above a predetermined threshold a drug in addition to the agonist is administered to treat the blood pressure.

39. The method of claim 38, wherein the threshold is a blood pressure of at least 145 systolic and/or 90 diastolic mm Hg.

40. The method of claim 38, wherein the patient has diabetes or proteinuria and the threshold is a blood pressure of at least 130 systolic and/or 80 diastolic mm Hg.

41. The method of claim 1, wherein the agonist is an agonist of an alpha-2A receptor.

42. The method of claim 1, wherein the agonist is an agonist of a d2 dopamine receptor.

43. A method of prophylaxis or treatment of hypertension in a patient having or at risk of hypertension, comprising:
administering an effective regime of an agonist of an α2A receptor and/or an agonist of a d2 receptor to a patient having a mutation in an α2C adrenergic receptor or a nucleic acid encoding the same, wherein the dosage is administered orally on a daily basis for at least a month, and the daily dosage is less than or equal to 10 mg/day.

48. A method of prophylaxis or treatment of cardiovascular disease in a patient having or at risk of the disease, comprising:
administering a daily dosage of less than 0.05 mg of clonidine for a period of at least a week to a patient to effect prophylaxis or treatment of the disease in the patient, wherein the patient has a mutation in an α2C adrenergic receptor or a nucleic acid encoding the same.
